(19) United States
(12) Patent Application Publication
(10) Pub. No.: US 2020/0146858 A1
(43) Pub. Date: May 14, 2020

(54) DUAL BALLOON CATHETERS AND METHODS FOR USE

(71) Applicant: Ostial Corporation, Campbell, CA (US)

(72) Inventors: Jake Wolenberg, Santa Clara, CA (US); Archimedes Sanati, Campbell, CA (US); Farhad Khosravi, Los Altos Hills, CA (US)

(21) Appl. No.: 16/679,118

(22) Filed: Nov. 8, 2019

Related U.S. Application Data
(60) Provisional application No. 62/757,688, filed on Nov. 8, 2018.

(51) Int. Cl. A61F 2/958

(52) U.S. Cl. CPC A61F 2/958; A61M 2025/1013

(57) ABSTRACT

A catheter is provided that includes first and second balloons on a catheter shaft. The first balloon includes a cylindrical main section extending between first and second ends thereof defining a first interior communicating with an inflation lumen of the catheter shaft, and a second balloon including a first end attached to the catheter shaft adjacent the first end of the first balloon and a second end extending over the main section of the first balloon such that the second balloon defines a second interior. The first balloon includes openings in the membrane of the first balloon that are located within the second interior to allow inflation media delivered through the inflation lumen to enter the first interior to inflate the first balloon and pass through the one or more openings into the second interior to inflate the second balloon sequentially or simultaneously with the first balloon.

The present application claims benefit of co-pending U.S. provisional application Ser. No. 62/757,688, filed Nov. 8, 2018, the entire disclosure of which is expressly incorporated by reference herein.

### FIELD OF THE INVENTION

The present invention relates generally to balloon catheters, and, more particularly, to catheters with dual balloons that may be inflated simultaneously or in rapid succession, e.g., for flaring or otherwise expanding stents or other prostheses deployed within a body lumen, dilating stenoses, and the like.

### BACKGROUND

Tubular endoprostheses or “stents” have been suggested for dilating or otherwise treating stenoses, occlusions, and/or other lesions within a patient's vasculature or other body lumens. For example, a self-expanding stent may be maintained on a catheter in a contracted condition, e.g., by an overlying sheath or other constraint, and delivered into a target location, e.g., a stenosis within a blood vessel or other body lumen. When the stent is positioned at the target location, the constraint may be removed, whereupon the stent may automatically expand to dilate or otherwise line the vessel at the target location.

Alternatively, a balloon-expandable stent may be carried on a catheter, e.g., crimped or otherwise secured over a balloon, in a contracted condition. When the stent is positioned at the target location, the balloon may be inflated to expand the stent and dilate the vessel.

Vascular stenoses, e.g., within arteries supplying the heart, can lead to hypoflow conditions in the vessel, which may cause ischemia and/or infarction of the organ being supplied by that vessel, e.g., the heart. Sometimes, a stenosis or other lesion may occur at an ostium or bifurcation, i.e., where a branch vessel extends from a main vessel or trunk. In such situations, it may be difficult to visualize the lesion and/or accurately position a stent within the ostium and/or branch vessel.

Often, when the stent is deployed in such anatomy, one end of the stent may extend out from the ostium into the main vessel, e.g., into the aorta from a coronary artery, common carotid artery, or peripheral artery. Such a position of the stent may cause difficulty in future endoluminal interventions, e.g., reentering the branch vessel, which may require recrossing the stent. In addition, there may be risk that the end of the stent extending from the ostium may cause damage to neighboring structures, such as aortic valve leaflets adjacent to the ostium of a coronary vessel. Furthermore, there may be little or no contact between the stent and the wall of the ostium, which may result in suboptimal treatment of the lesion.

To address these problems, it has been suggested to over-expand or flare the end of the stent extending into the main vessel, e.g., to direct the end of the stent against or closer to the wall of the ostium. Thereafter, it may be easier to recross the stent in case future endovascular study or intervention is required. In addition, in the case of coronary stents, flaring the end of the stent extending into the aorta may also move the end further from the aortic lumen and decrease the risk of damage to the aortic leaflets. Furthermore, direct contact or closer positioning of the stent to the wall of the ostium may result in improved treatment of the lesion.

Accordingly, apparatus and methods for flaring and/or otherwise expanding stents or other prostheses would be useful.

### SUMMARY

The present invention is directed to balloon catheters, and, more particularly, to catheters including dual balloons that may be inflated simultaneously or in rapid succession, e.g., for flaring or otherwise expanding stents or other prostheses deployed within a body lumen, dilating stenoses, and the like.

In accordance with an exemplary embodiment, an apparatus is provided for performing a medical procedure that includes an elongate tubular member comprising a proximal end, a distal end sized for introduction into a patient's body, and an inflation lumen extending between the proximal and distal ends; and first and second balloons on the distal end that may be inflated sequentially or simultaneously. For example, the first balloon may include first and second ends attached to the distal end at spaced apart locations and a substantially cylindrical main section extending between the first and second ends such that the first balloon defines a first interior communicating with the inflation lumen; the second balloon may include a first end attached to the distal end adjacent the first end of the first balloon and a second end extending at least partially over the main section of the first balloon such that the second balloon defines a second interior. The first balloon may include one or more openings in a membrane of the first balloon adjacent the first end, e.g., a plurality of holes or slits, such that the one or more openings are located within the second interior to allow inflation media delivered through the inflation lumen to enter the first interior to inflate the first balloon and pass through the one or more openings into the second interior to inflate the second balloon sequentially or simultaneously with the first balloon.

In accordance with another embodiment, a method is provided for flaring a stent previously deployed within a branch body lumen including an ostium communicating with a main body lumen, a first end of the stent extending at least partially from the branch body lumen into the ostium. The method may include providing an elongate member including a proximal end, a distal end, a first balloon carried on the distal end that includes a substantially uniform diameter main section when expanded, and a second balloon carried on the distal end that includes a distal section overlying at least a portion of the main section of the first balloon and a proximal section extending proximally from the distal section and the first balloon. The distal end may be introduced into the main body lumen, e.g., with the balloons collapsed and the distal end may be positioned through the ostium and stent into the branch body lumen until the main section is disposed within the stent. Inflation media may be delivered through a lumen into an interior of the first balloon such that the first balloon at least partially inflates to substantially anchor the stent axially relative to the branch body lumen, and at least some of the inflation media passes through one or more openings in a membrane of the first balloon into an interior of the second balloon to inflate the second balloon to flare the first end of the stent within the ostium.

Other aspects and features of the present invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1A is a side view of an exemplary embodiment of a catheter including dual balloons for expanding a prosthesis or dilating a stenosis within a body lumen.

FIG. 1B is a cross-sectional detail of a distal end of the catheter of FIG. 1A with the balloons expanded.

FIGS. 2A and 2B are details showing exemplary embodiments of inner balloons that may be provided on the catheter shown in FIGS. 1A and 1B.

FIGS. 3A-3E are cross-sectional views of a patient's body including an ostium communicating between a main vessel and a branch vessel, showing a method for flaring a stent previously deployed within the branch vessel using the catheter of FIGS. 1A and 1B.

### DETAILED DESCRIPTION OF THE EXEMPLARY EMBODIMENTS

Before the exemplary embodiments are described, it is to be understood that the invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

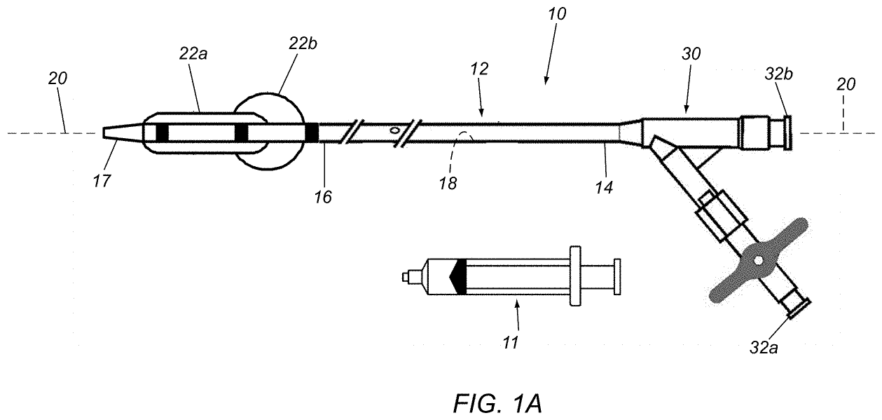

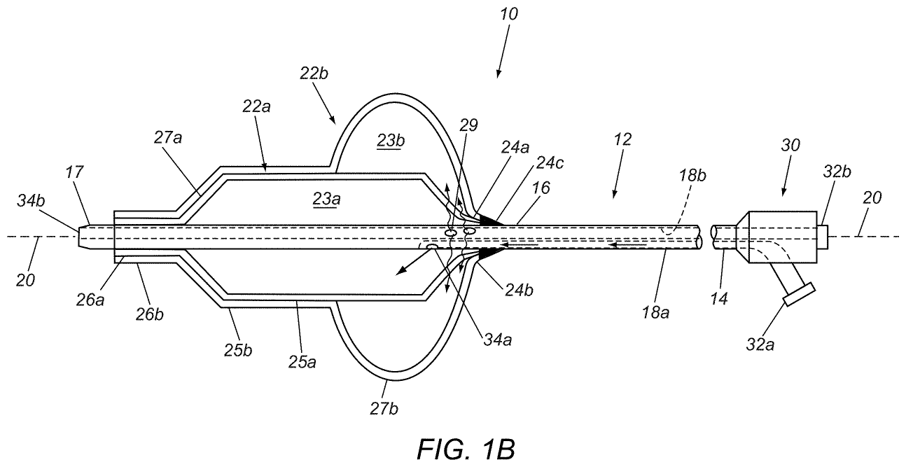

Turning to the drawings, FIGS. 1A and 1B show an exemplary embodiment of a balloon catheter or apparatus 10 includes an elongate tubular member or shaft 12 having a proximal end 14, a distal end 16, and one or more lumens 18 extending between the proximal and distal ends 14, 16, thereby defining a longitudinal axis 20 extending between the proximal and distal ends 14, 16. Optionally, the catheter 10 may be provided as part of a kit or system including one or more additional components, such as one or more sources of inflation media or vacuum, e.g., syringe 11 (shown in FIG. 1A), a stent carried on the apparatus 10, a guide catheter, and/or one or more guidewires (not shown).

As shown, the catheter 10 may include a pair of overlapping balloons or other expandable members 22 on the distal end 16, e.g., a first or distal balloon 22a, and a second or proximal balloon 22b at least partially overlying the first balloon 22a. The distal end 16 of the catheter 10 (and consequently, the balloons 22) may be introduced into a patient's body for performing one or more medical procedures, e.g., for flaring and/or otherwise expanding a stent previously deployed within a body lumen, for delivering a stent carried on the distal end 16, for dilating a stenosis or valve, and/or for performing one or more other procedures within a patient's body (not shown), e.g., similar to the methods described elsewhere herein and in U.S. Publication Nos. 2006/0265041, 2007/0073388, and 2013/0060316, the disclosures of which are expressly incorporated by reference herein.

In addition, the distal end 16 may include one or more markers, e.g., one or more bands of radiopaque material 19 as shown in FIG. 1A, for imaging the distal end 16 using external imaging, e.g., fluoroscopy and the like, to facilitate positioning the catheter 10 relative to a stent and/or anatomical structures within a patient's body. In addition or alternatively, the catheter 10 may include one or more therapeutic and/or diagnostic elements (not shown) on the distal end 16, e.g., within or carried by the balloon(s) 22, as described further below.

The shaft 12 may be formed from one or more tubular bodies, e.g., having variable flexibility along its length. For example, the distal end 16 may be substantially flexible to facilitate introduction through tortuous anatomy, e.g., terminating in a rounded, tapered, and/or other substantially atraumatic distal tip 17. The distal end 16 may be sized and/or shaped for introduction into a body lumen, e.g., having a diameter between about one and seven millimeters (1.0-7.0 mm), or less than 1.7 millimeters. The proximal end 14 may be substantially flexible, semi-rigid, or rigid, e.g., having sufficient column strength to facilitate advancing the distal end 16 through a patient's vasculature by pushing on the proximal end 14 without buckling or kinking. Optionally, a shaft support wire or other stiffener (not shown) may be provided within the proximal end 14, if desired, e.g., to facilitate pushing the catheter 10 from the proximal end 14. The shaft 12 may be formed from plastic, metal, or composite materials, e.g., a plastic material having a wire, braid, or coil core, which may prevent kinking or buckling of the shaft 12 during advancement and/or other manipulation.

As shown in FIG. 1A, the catheter 10 may include a handle or hub 30 on the proximal end 14, e.g., to facilitate manipulating the catheter 10. The handle 30 may include one or more ports 32 communicating with respective lumens 18 within the tubular member 12, as described further below. The handle 30 may be molded, machined, or otherwise formed from plastic, metal, or composite material, e.g., providing an outer casing, which may be contoured or otherwise shaped to ease manipulation. The proximal end 14 of the tubular member 12 may be attached to the handle 30, e.g., by bonding, cooperating connectors, interference fit, and the like. Optionally, if the catheter 10 includes any actuatable components (not shown) on the distal end 16, the handle 30 may include one or more actuators (also not shown), such as one or more slides, dials, buttons, and the like, for activating, actuating, or otherwise manipulating the components from the proximal end 14.

In the exemplary embodiment shown in FIG. 1B, the tubular member 12 includes at least two lumens 18 extending between the proximal and distal ends 14, 16. For example, the tubular member 12 may include an inflation lumen 18a that extends from port 32a in the handle 30 through the tubular member 12 to one or more openings, e.g., opening 34a, thereby communicating directly within an interior 23a of the first balloon 22a and indirectly with an interior 23b of the second balloon 22b, as described further below. The port 32a may include one or more connectors, e.g., a Luer lock connector (not shown), one or more seals (also not shown), and the like, to facilitate coupling an inflation device, e.g., a syringe 11, to the handle 30.

In addition, the shaft 12 may include an instrument lumen 18b that extends from a port 32b in the handle 30 to an opening 34b in the distal tip 17. The instrument lumen 18b may have sufficient size to allow a guidewire or other rail or instrument (not shown) to be inserted therethrough, e.g., to facilitate advancing the catheter 10 over the rail, as explained further below. Optionally, the port 32b may include one or more seals (not shown) that prevent fluid, e.g., blood, from flowing proximally out of the port 32b, yet allow one or more instruments to be inserted therethrough and into the instrument lumen 18b. Alternatively, a “rapid exchange” instrument lumen (not shown) may be provided that extends from a proximal port on the shaft 12, e.g., offset proximally a desired distance from the distal end 16, to the opening 34b instead of instrument lumen 18b.

Returning to FIGS. 1A and 1B, the shaft 12 includes a first or inner balloon 22a and a second or outer balloon 22b on the distal end 16, which may be bonded or otherwise secured to the distal end 16 of the shaft 12 and/or to each other, e.g., by bonding with adhesive, sonic welding, using an annular collar or sleeve, and the like. For example, as best seen in FIG. 1B, the inner balloon 22a may include a proximal end 24a attached directly to the distal end 16 and a distal end 26a attached directly to the distal end 16 adjacent the distal tip 17. The inner balloon 22a may include a central or main section 25a, e.g., having a cylindrical shape defining a substantially uniform diameter, and tapered end regions that taper from the main section 25a to the proximal and distal ends 24a, 26a.

The outer balloon 22b also includes a proximal end 24b that may be attached to the distal end 16 adjacent to the proximal end 24a of the first balloon 22b, e.g., over or proximal to the proximal end 24a of the first balloon 22b, and a distal end 26b that may be attached to the distal end 16 or to the first balloon 22a. For example, as shown in FIG. 1B, both proximal ends 24a, 24b of the inner/outer balloons 22a, 22b may include tubular legs that overlap such that they are attached to the distal end 16 at the same location, e.g., by bonding with adhesive, sonic welding, and/or using an annular collar or sleeve 24c.

The outer balloon 22b may extend over the inner balloon 22a such that the distal ends 26a, 26b also overlap and are attached at the same location to the distal end 16, e.g., adjacent the distal tip 17. Alternatively, the distal end 26b of the outer balloon 22b may be attached to the inner balloon 22a at a location proximal to the distal end 26a, e.g., to the main section 25a and/or other location, e.g., similar to embodiments described in the publications incorporated by reference herein.

In addition, the outer balloon 22b may include a first or distal section 25b that extends at least partially over the inner balloon 22a and a second or proximal section 27b disposed around or adjacent the proximal end 24a of the inner balloon 22a. For example, as shown in FIG. 1B, the first section 25b may extend entirely over the inner balloon 22a and the distal end 26b of the outer balloon 22b may be attached over or adjacent to the distal end 26a of the inner balloon 22a. The first section 25b of the outer balloon 22b may overlie but remain separate from the underlying inner balloon 22a. Alternatively, the first section 25b may be bonded or otherwise attached to the inner balloon 22a, e.g., continuously or intermittently along the inner balloon 22a.

The distal section 25b may have a cylindrical shape defining a substantially uniform diameter, e.g., similar to the main section 25a of the inner balloon including a tapered end region transitioning to the distal end 26b. The proximal section 27b of the outer balloon 22b may have a substantially spherical or other bulbous shape when expanded, e.g., having a diameter that is larger than the diameter of the distal section 25b, as described further below. Alternatively, the distal section 25b may be omitted and a distal end of the proximal section 27b may be attached to the inner balloon 22a, e.g., around the main section 25a (not shown).

Optionally, the orientation of the outer balloon 22b may be reversed, if desired, e.g., with the second section 25b of the outer balloon 22b extending distally relative to the main section 25a of the inner balloon 22a rather than proximally. In a further alternative, a substantially spherical or bulbous section may be provided on the outer balloon 22b both proximally and distally to the main section 25a of the inner balloon 22a (not shown).

The inner balloon 22a may be expandable from a contracted condition (not shown), e.g., folded, rolled, or otherwise positioned closely around the distal end 16 for delivery, to an enlarged condition (shown in FIGS. 1A and 1B). Similarly, the outer balloon 22b may also be expandable from a contracted condition (not shown) to an enlarged condition (shown in FIGS. 1A and 1B). One or both balloons 22 may be formed from substantially inelastic material, e.g., PET, nylon, or PEBAX, such that the balloon(s) 22 expands to a predetermined size in its enlarged condition once sufficient fluid is introduced into the interior of the balloon 622. In addition or alternatively, one or both balloons 22, e.g., outer balloon 22b, may be formed from substantially elastic material, e.g., silicone, polyurethane, or polyethylene, such that the balloon(s) 22 may be expanded to a variety of sizes depending upon the volume and/or pressure of fluid within the interior. In an exemplary embodiment, the inner balloon 22a may be formed from a semi-compliant or non-compliant material, e.g., mid to high durometer PEBAX, nylon, or PET, and the outer balloon 22b may be formed from a substantially compliant or semi-compliant material, e.g., polyethylene, polyurethane, and low to mid durometer PEBAX, i.e., having a higher compliance than the inner balloon 22a. For example, using more compliant material for the outer balloon may improve safety by ensuring that the outer balloon will burst at a lower pressure/volume, e.g., before it reaches an unsafe diameter or pressure. More compliant/different material may also aid in reducing the wall thickness/folded diameter of the outer balloon 22b and/or reduce sticking between the inner and outer balloon materials.

Alternatively, the inner and outer balloons 22a, 22b may be formed from the same material, e.g., having the same thickness and/or mechanical properties. Using material with the same compliance for both the outer balloon 22b and the inner balloon 22a, e.g., inelastic material to provide non-compliant balloons, may allow for both balloons to be used for higher pressure dilatations than may be achieved using a compliant outer balloon. Such higher pressures may be useful for expanding calcified lesions or tough plaques.

To provide the proximal and distal sections 27b, 25b of the outer balloon 22b, the balloon material may be formed into a shape including a substantially spherical or other bulbous shape for the proximal section 27b and a substantially uniform, smaller diameter shape for the distal section 25b. For example, the balloon material may be blow molded within a mold (not shown) having the desired shape for the outer balloon 22b when inflated. If the outer balloon 22b is formed from compliant material, the proximal section 27b, may be expanded greater than the relaxed molded shape, yet may substantially maintain the bulbous shape unless constrained by external forces.

The outer balloon 22b may have a substantially uniform wall thickness, e.g., between the proximal and distal sections 27b, 25b. Alternatively, the wall thickness may vary; for example, the proximal section 27b may have a thinner wall thickness than the distal section 25b. Optionally, the outer balloon 22b may include one or more features thereon for enhancing traction, friction, or other engagement with structure contacted by the outer balloon 22b when expanded. For example, the outer surface of at least the proximal section 27b may be treated or textured, may include ribs or other protrusions, and the like (not shown) to increase friction or other engagement upon expansion.

In addition or alternatively, the balloons 22 may operate under different internal pressures and/or may require different pressures sufficient to fully expand the respective balloons 22. For example, the inner balloon 22a may require a greater inflation pressure to fully expand than the outer balloon 22b. This may allow the proximal section 27b of the outer balloon 22b to be expanded using a lower inflation pressure to flare and/or shape a flaring portion of a stent without substantial expansion of a main portion of the stent, as described further elsewhere herein and in the publications incorporated by reference herein.

As shown in FIGS. 1A and 1B, the proximal section 27b of the outer balloon 22b may be shaped to expand to a substantially spherical shape in the enlarged condition, e.g., having a diameter between about ten and twenty millimeters (10-20 mm) when expanded using an inflation pressure between about one and five atmospheres (1-5 ATM). In an exemplary embodiment, the proximal section 27b of the outer balloon 22b may have a diameter of about thirteen millimeters (13 mm) at an inflation pressure of about two atmospheres (2 ATM). In contrast, the inner balloon 122b may be shaped to expand to a substantially cylindrical shape in the enlarged condition, e.g., having a diameter between about two and eight millimeters (2-8 mm) when expanded using an inflation pressure between about eight and twenty atmospheres (8-20 ATM).

In addition, the main section 25a of the inner balloon 22a may have a substantially uniform diameter, e.g., having a length between about eight and thirty millimeters (8-30 mm). Beyond the uniform diameter portion, the inner balloon 22a may have a transition portion 27a adjacent the distal tip 17. The transition portion 27a may be tapered, as shown, or may be substantially blunt, i.e., extending inwardly to the distal tip 17 (not shown). As shown, the main portion 25a of the inner balloon 22a may underlie at least a portion of the outer balloon 22b, e.g., the distal section 25b, as shown in FIG. 1B and as disclosed in the publications incorporated by reference herein. In an exemplary embodiment, the main section 27a of the inner balloon 22a may have a diameter of between about five and six millimeters (5-6 mm) in the enlarged condition and may have a length of at least about seventeen millimeters (17 mm) distally beyond the proximal section 27b of the outer balloon 22b. Additional information regarding exemplary balloons and/or methods for making balloons may be found in the publications incorporated by reference herein.

Returning to FIG. 1B, the inner balloon 22a defines a substantially enclosed interior 23a between the proximal and distal ends 24a, 26 that communicates with the inflation lumen 18a. For example, as shown, one or more inflation ports, e.g., port 34a, may be provided in the distal end 16 within the interior 23a that communicates with the inflation lumen 18a. Thus, fluid or other inflation media delivered through the inflation lumen 18a may exit the inflation port 34a and inflate the inner balloon 22a, and conversely, vacuum applied to the inflation lumen 18a may be applied to the first interior 23a to collapse the inner balloon 22a.

In addition, the inner balloon includes one or more openings, e.g., a plurality of openings 29, as shown in FIG. 1B, formed in the balloon membrane of the inner balloon 22a adjacent the proximal end 24a to allow at least some fluid delivered through the inflation lumen 18a to pass through the openings 29 into an interior 23b of the outer balloon 22b in a desired manner, e.g., as represented by the arrows in FIG. 1B, to inflate at least the proximal section 27b of the outer balloon 22b sequentially or simultaneously with the inner balloon 22a.

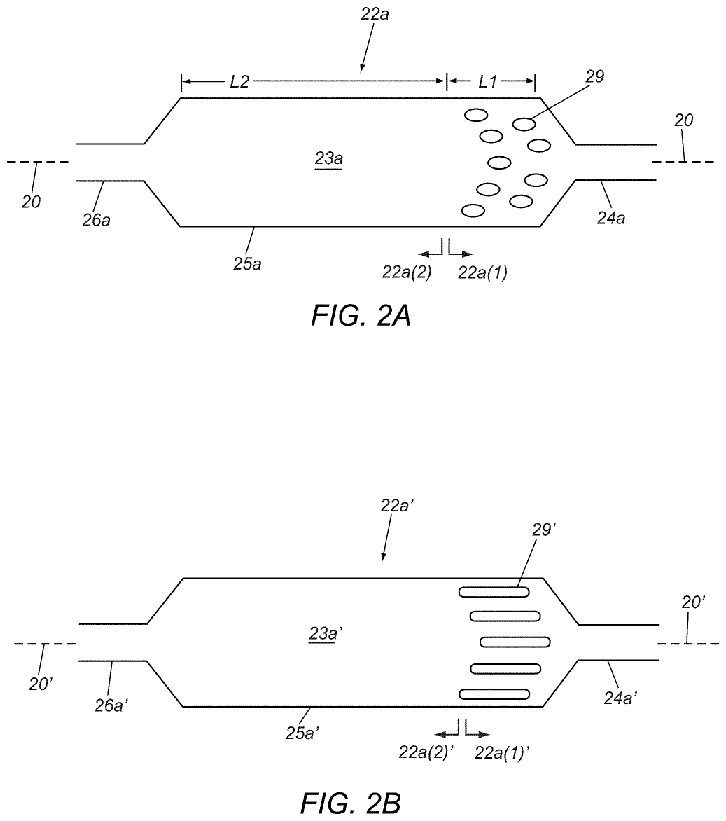

For example, as shown in FIG. 2A, an inner balloon 22a is shown that includes a plurality of openings 29 spaced apart from one another around a circumference of a proximal or “holey” region 22a(1) of the inner balloon 29, e.g., disposed adjacent the first end 24a. Thus, the inner balloon 22a also includes a distal region 22a(2) extending from the proximal region 22a(1) to the second end 26a of the inner balloon 22a without any openings in the membrane, i.e., to provide an “intact” region. In an exemplary embodiment, the proximal region 22a(1) has a length L1 along the longitudinal axis 20 that is shorter than a length L2 of the distal region 22(2), e.g., such that the proximal region 22a(1) is disposed within the interior 23b of the proximal region 27b of the outer balloon 22b and the distal region 22a(2) extends along the distal portion 25b of the outer balloon 22b, e.g., as shown in FIG. 1B.

In the exemplary embodiment shown in FIG. 2A, each of the openings 29 may have an elliptical shape and may be arranged in one or more circumferential sets, e.g., two circumferential sets disposed axially adjacent one another as shown. Alternatively, the openings 29 may have circular or other shapes and/or may be distributed in other configurations around or on the proximal region 22a(1). For example, as shown in FIG. 2B, an inner balloon 22′ is shown that includes a circumferential set of slits 29′ formed in the balloon membrane, e.g., with the lengths of the slits aligned substantially parallel to the longitudinal axis 20.′

Optionally, openings may be provided in the tapered end region tapering to the first end 24a, e.g., in addition to or instead of the openings 29 in the proximal region 22a(1) of the main section 25a. In another alternative, one or more openings may be provided in the first end 24a, e.g., in a tubular leg defining the first end 24a in addition to or instead of the openings 29. This alternative may minimize risk of the openings 29 being blocked when the balloon 22a is rolled or folded around the distal end 16 into a contracted configuration, e.g., for introduction into a patient's body.

The size and number of the openings 29, as well as the material and/or other mechanical properties of the membrane of the inner balloon 22a may be selected to provide a desired flow rate of fluid through the openings 29 into the outer balloon 22b. For example, if the inner balloon 22a is formed from non-compliant or inelastic material, the size of the openings 29 may remain unchanged as the inner balloon 22a inflates or deflates. Alternatively, the material may have sufficient elasticity to dilate the openings 29 at a desired pressure, e.g., to allow the inner balloon 22a to at least partially inflate before fluid passes through the dilating openings 29 into the outer balloon 22b, as described further below. In an exemplary embodiment, the size and number of the openings 29 may be selected to allow the main section 25 of the inner balloon 22a to initially expand to its cylindrical shape before half the fluid needed to inflate the proximal portion 27b of the outer balloon 22b has passed through the openings 29.

The openings 29 may be formed in the material of the inner balloon 22a using a variety of methods. For example, the openings 29 may be formed using a heated pin, blade, stamp, and the like, or may be laser cut into the material, e.g., after molding or otherwise forming the inner balloon 22a. Alternatively, the openings 29 may be formed simultaneously with molding or otherwise forming the inner balloon 22a, e.g., using one or more mandrels or other components positioned within a mold cavity when the balloon is formed.

Returning to FIGS. 1A and 1B, optionally, the catheter 10 may include one or more additional features. For example, in one option, the catheter shaft 12 may include an additional lumen that communicates with the interior 23b of the outer balloon 22b independent of the openings 29. Such a lumen may allow a vacuum to be applied directly to the interior 23b, e.g., to facilitate collapsing the outer balloon 22b independent of the inner balloon 22a, i.e., rather than relying on a vacuum applied to the inflation lumen 18a reaching the interior 23b through the openings 29 (which may become blocked as the inner balloon 22a is folded or collapsed by the vacuum).

In addition or alternatively, a pressure relief valve or other feature (not shown) may be provided on the proximal end 14 of the shaft 12 and/or on the handle 30. The pressure relief feature may be coupled to or communicate with the inflation lumen 18a to prevent unintentional over-inflation of the balloons 22. For example, if syringe 11 (or other source of inflation media) is connected to the port 32a and actuated, fluid or other inflation media may pass through the inflation lumen 18a into the interior 23a of the inner balloon 22a and then pass through the openings 29 into the interior 23b of the outer balloon 22b. If the pressure and/or volumetric flow rate of the fluid exceeds a maximum threshold, the pressure relief feature may open to release the pressure and/or allow fluid to escape, e.g., at the handle 30 and prevent the balloons 22a, 22b from over-inflating, rupturing, and/or otherwise failing during use.

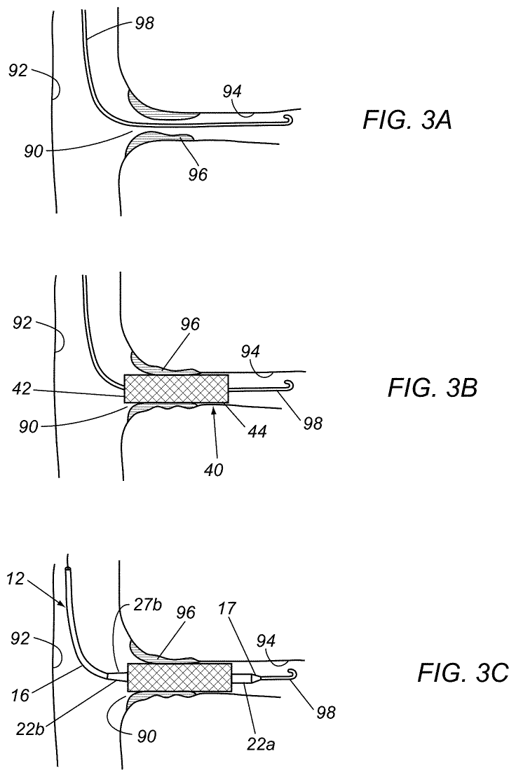

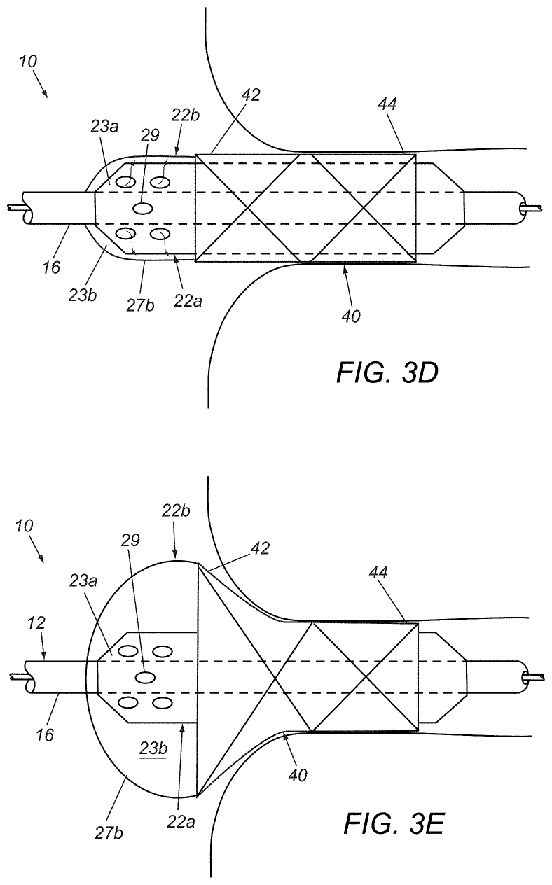

Turning to FIGS. 3A-3E, an exemplary method is shown for using the catheter 10 to flare and/or otherwise expand a stent 40 deployed within a patient's body, e.g., including an ostium 90. As shown, the ostium 90 may be an opening in a wall of a first or main body lumen or trunk 92 that communicates with a second body lumen or branch 94. In exemplary embodiments, the main body lumen 92 may be the ascending or descending aorta, and the branch body lumen may be a coronary artery, a common carotid artery, or a peripheral artery. A stenosis, occlusion, or other lesion 96 may exist at and/or adjacent to the ostium 90, e.g., extending at least partially into the branch 94. The lesion 96 may include atherosclerotic plaque or other material that partially or completely occludes blood or other fluid flow between the trunk 92 and the branch 94.

Initially, as shown in FIG. 3A, a guidewire 98 or other rail may be introduced from the trunk 92 through the ostium 90 into the branch 94, e.g., using conventional methods. For example, a percutaneous puncture or cut-down may be created at a peripheral location (not shown), such as a femoral artery, carotid artery, or other entry site, and the guidewire 98 may be advanced through the patient's vasculature from the entry site, e.g., alone or with the aid of a guide catheter (not shown). For example, a distal end of a guide catheter (not shown) may be advanced over the guidewire 98 into the trunk 92, e.g., until the distal end is disposed adjacent or proximal to the ostium 90. The guide catheter may be used to advance one or more instruments (such as any of the catheters or other devices described herein) over the guidewire 98 and into the trunk 92 and/or branch 94.

If the lesion 96 completely occludes the branch 94, the guidewire 98 may be directed through the occlusion, or other devices (not shown) may be advanced over the guidewire 98 or otherwise in conjunction with the guidewire 98 to create a passage through the lesion 96 for the guidewire 98.

After the guidewire 98 is directed into the branch 94 beyond the lesion 96, it may be desirable to at least partially dilate the lesion 96. For example, an angioplasty catheter (not shown) may be advanced through the guide catheter and/or over the guidewire 98 into and through the lesion 96, whereupon a balloon or other element on the catheter may be expanded to at least partially dilate the lesion 96. If desired, other procedures may also be performed at the lesion 96, e.g., to soften, remove, or otherwise treat plaque or other material forming the lesion 96, before the stent 40 is implanted. After completing any such procedures, any instruments advanced over the guidewire 98 may be removed.

To deliver the stent 40, any delivery catheter and/or conventional procedure may be used. For example, a distal end of a delivery catheter (not shown) may be advanced over the guidewire 98 and/or through the guide catheter from the entry site into the trunk 92. For example, with the distal end of the guide catheter against or adjacent the ostium 90, the distal end of the delivery catheter may be advanced from the guide catheter, through the ostium 90, and into the branch 94. The delivery catheter may be positioned such that the stent 40 extends into and through the lesion 96 and/or branch 94. The stent 40 may be expanded and/or otherwise deployed from the delivery catheter to place the stent 40 across the lesion 96 and/or within the branch 94. For example, as shown in FIG. 3B, the stent 40 may be deployed such that a first end 42 of the stent 40 extends at least partially into the ostium 90 and/or the trunk 92, and a second end 44 of the stent 40 is disposed within the branch 94 beyond the lesion 96.

As shown, the stent 40 may have a substantially uniform diameter cross-section once deployed. For example, the stent 40 may be expanded to dilate and/or otherwise engage the lesion 96 and/or branch 94. Alternatively, the stent 40 may be partially expanded using the delivery catheter, allowing the stent 40 to be further expanded by the apparatus 10, as described below.

Turning to FIG. 3C, thereafter, the distal end 16 of the catheter 10 (with the balloons 22 in their contracted conditions) may be introduced into the trunk 92 to flare and/or otherwise expand the stent 40. Before introduction, a source of vacuum, e.g., syringe 11 or an indeflator (not shown) may be coupled to the port 32a (shown in FIG. 1A) and actuated to apply a vacuum to simultaneously collapse and/or otherwise prepare the balloons 22 for introduction. The balloons 22 may then be rolled, folded, or otherwise constrained in a contracted condition for introduction into the patient's body. In the embodiment shown in FIG. 1B, once the catheter 10 is ready for introduction, a proximal end (not shown) of the guidewire 98 may be backloaded into the opening 34b through the instrument lumen 18b and out the port 32b. The distal end 16 may then be advanced over the guidewire 98 into the patient's body over the guidewire 98.

As shown in FIG. 3C, the distal end 16 may be advanced through the stent 40 and ostium 90 at least partially into the branch 94. For example, the distal end 16 may be positioned such that the inner balloon 22a is positioned within and/or beyond the stent 40, e.g., beyond the first end 42, and the proximal section 27b of the outer balloon 22b is positioned adjacent the ostium 90, e.g., within and/or proximal to the first end 42 of the stent.

Optionally, to facilitate positioning, the distal end 16 may be monitored using fluoroscopy or other external imaging, e.g., to observe and monitor markers 19 (not shown, see FIG. 1B) on the distal end 16. For example, markers 19a and 19b may be located on the distal end 16 to identify the ends of the substantially uniform main section 25a of the inner balloon 22a, while proximal marker 19c may be located on the distal end 16 to identify the proximal end 24b and/or proximal section 27b of the outer balloon 22b. Thus, with the markers 19, the inner balloon 22a may be aligned with the distal end 44 and/or portion of the stent 40 within the branch 94 beyond the ostium 90 and the proximal section 27b of the outer balloon 22b may be aligned with the first end 42 of the stent 40 and/or the ostium 90, as desired.

Turning to FIG. 3D, with the distal end 16 positioned as desired, the syringe 11 or other source of inflation media (not shown) may be operated to inflate the balloons 22 in a desired manner to flare and/or otherwise further expand the stent 40, e.g., in a single continuous action using a single source of inflation media. For example, as shown, as fluid initially flows into the interior 23a of the inner balloon 22a, the inner balloon 22a may inflate to engage the stent 40 and/or wall of the branch 94, e.g., to prevent substantial axial migration of the stent 40.

As fluid continues to be delivered, the fluid may pass through the openings 29 into the interior 23b of the outer balloon 22b, thereby inflating the proximal section 27b of the outer balloon 22b to flare the stent 40 while the inner balloon 22a prevents migration, e.g., as shown in FIG. 3E. Optionally, the openings 29 may be configured such that fluid flows simultaneously into the outer balloon 22b to flare the first end 42 and further into the inner balloon 22a to further dilate the stent 40 within the branch 94, as shown in FIG. 3E. For example, the size and number of openings 29 may cause fluid to pass into outer balloon 22b at no more than half the flow rate entering the inner balloon 22a, e.g., to inflate the balloons 22a simultaneously but expanding the outer balloon 22b more slowly than the inner balloon 22a to ensure engagement of the inner balloon 22a with the stent 40. Thus, the first end 42 of the stent 40 may be expanded into a flared configuration, which may conform to the shape of the proximal section 27b and/or the ostium 90. With the inner balloon 22a expanded, the stent 40 and distal end 16 may remain substantially stationary during this inflation and flaring.

If a pressure relief feature is provided, the user may rapidly actuate the syringe 11 in a single motion, which may reduce inflation time for the balloons 22 while minimizing the risk of rupture or other damage to the balloons 22. If the pressure or flow rate of fluid delivery exceeds a predetermined threshold, the pressure relief feature may automatically activate, e.g., opening a valve in the handle 30 to release excess fluid and/or pressure, e.g., to set a maximum inflation pressure and/or rate of the balloons 22.

Once the stent 40 is dilated and/or flared as desired, the balloons 22 may be deflated or otherwise collapsed, e.g., simultaneously, by applying a vacuum to the inflation lumen 18a to withdraw fluid from the interior 23a of the inner balloon 22a via the port 34a and from the interior 23b of the outer balloon 22b via the openings 29 (or, alternatively, via a separate deflation lumen communicating with the interior 23b of the outer balloon 22b). The catheter 10 may then be withdrawn from the branch 94 and trunk 92, and from the patient's body, e.g., into the guide catheter (not shown). The guide catheter and/or guidewire 98 may then be removed from the patient's body, leaving the stent 40 in place.

If the catheter 10 is used only to flare a stent 40 already deployed and expanded within the branch 94, the inner balloon 22a does not need to be used for high pressure dilation of the stent 40. In this option, the wall thickness of the inner balloon membrane may be decreased to reduce the profile of the distal end 16 during introduction and/or enhance trackability of the distal end 16.

In an alternative embodiment, the catheter 10 may be used to deliver and dilate a stent, e.g., stent 40 into ostium 90, carried on the distal end 16 over the balloons 22. For example, the first end 42 of the stent 40 may be positioned over or adjacent the proximal region 27b of the outer balloon 22a and the remainder of the stent 40 may be positioned over the main section 25a of the inner balloon 22a (and consequently over the distal region 25b of the outer balloon 22b). Once the stent is positioned as desired within the ostium 90, e.g., similar to the position shown in FIGS. 3B-3D, the balloons 22 may be inflated to expand the stent 40 and flare the first end 42, e.g., simultaneously or in rapid succession.

It will be appreciated that the catheters and balloons described herein may provide one or more advantages over catheters that include separate inflation lumens, i.e., that would otherwise allow the inner and outer balloons to be inflated and/or deflated independently of one another. For example, rather than providing two separate, relatively small inflation lumens running the length of the catheter shaft, a single, relatively large inflation lumen may be provided within the shaft. Such a relatively large inflation lumen may reduce drag and/or other wall effects, which may decrease inflation times (particularly if a pressure relief feature is included) and/or decrease deflation times during use. In addition, the catheters may facilitate manipulation and/or operation by a user since only a single inflation/deflation source is needed, eliminating the need to coordinate multiple sources, e.g., remembering which syringe is communicating with which balloon.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. An apparatus for performing a medical procedure, comprising:
an elongate tubular member comprising a proximal end, a distal end sized for introduction into a patient's body, a longitudinal axis extending between the proximal and distal ends, and an inflation lumen extending between the proximal and distal ends;
a first balloon comprising first and second ends attached to the distal end at spaced apart locations and a substantially cylindrical main section extending between the first and second ends such that the first balloon defines a first interior communicating with the inflation lumen; and
a second balloon on the distal end comprising a first end attached to the distal end adjacent the first end of the first balloon and a second end extending at least partially over the main section of the first balloon such that the second balloon defines a second interior,
wherein the first balloon comprises one or more openings in a membrane of the first balloon adjacent the first end such that the one or more openings are located within the second interior to allow inflation media delivered through the inflation lumen to enter the first interior to inflate the first balloon and pass through the one or more openings into the second interior to inflate the second balloon sequentially or simultaneously with the first balloon.

2. The apparatus of claim 1, wherein the one or more openings comprise a plurality of openings spaced apart from one another around a circumference of a proximal region of the first balloon.

3. The apparatus of claim 2, wherein the first balloon further comprises a distal region extending from the proximal region to the second end of the first balloon without any openings in the membrane.

4. The apparatus of claim 3, wherein the proximal region has a length along the longitudinal axis that is shorter than a length of the distal region.

5. The apparatus of claim 2, wherein the plurality of openings comprise one or more of circular and elliptical holes formed through the membrane.

6. The apparatus of claim 2, wherein the plurality of openings comprise elongate slits formed through the membrane.

7. The apparatus of claim 1, wherein the first balloon is formed from non-compliant or semi-compliant material.

8. The apparatus of claim 7, wherein the second balloon is formed from material having a greater compliance than the first balloon material.

9. The apparatus of claim 7, wherein the second balloon is formed from elastic material.

10. The apparatus of claim 1, wherein the second balloon is formed from material having a predetermined shape comprising a first region defining a bulbous shape adjacent the first end of the second balloon and a second region extending from the first region to the second end defining a substantially cylindrical shape.

11. The apparatus of claim 1, wherein the distal end of the tubular member comprises one or more ports communicating with the inflation lumen and wherein the one or more openings are axially aligned with the one or more ports.

12. The apparatus of claim 1, further comprising a pressure relief feature communicating with the inflation lumen, the pressure relief feature configured to release at a predetermined threshold to prevent over-inflation of one or both of the inner and outer balloons.

13. The apparatus of claim 12, wherein the pressure relief feature comprises a valve at the proximal end that opens when pressure or flow rate within the inflation lumen exceeds the predetermined threshold.

14-18. (canceled)

19. A system for performing a medical procedure, comprising:
a) a catheter comprising:
i) an elongate tubular member comprising a proximal end including a handle or hub, a distal end sized for introduction into a patient's body, a longitudinal axis extending between the proximal and distal ends, and an inflation lumen extending from a port on the handle or hub to inflation port on the distal end;
ii) a first balloon comprising first and second ends attached to the distal end at spaced apart locations and a substantially cylindrical main section extending between the first and second ends such that the first balloon defines a first interior communicating with the inflation port;
iii) a second balloon on the distal end comprising a first end attached to the distal end adjacent the first end of the first balloon and a second end extending at least partially over the main section of the first balloon such that the second balloon defines a second interior; and
iv) a pressure relief feature on the handle or hub communicating with the inflation lumen to limit pressure or flow rate within the inflation lumen to a predetermined limit; and
b) a source of inflation media connectable to the port on the hub for delivering inflation media into the inflation lumen,
wherein the first balloon comprises one or more openings in a membrane of the first balloon adjacent the first end such that the one or more openings are located within the second interior to allow inflation media delivered through the inflation lumen to enter the first interior to inflate the first balloon and pass through the one or more openings into the second interior to inflate the second balloon sequentially or simultaneously with the first balloon.

20. A method for flaring a stent previously deployed within a branch body lumen including an ostium communicating with a main body lumen, a first end of the stent extending at least partially from the branch body lumen into the ostium, the method comprising:
providing an elongate member including a proximal end, a distal end, a first balloon carried on the distal end that includes a substantially uniform diameter main section when expanded, and a second balloon carried on the distal end that includes a distal section overlying at least a portion of the main section of the first balloon and a proximal section extending proximally from the distal section;
introducing the distal end into the main body lumen;
positioning the distal end through the ostium and stent into the branch body lumen until the main section is disposed within the stent; and
delivering inflation media through a lumen into an interior of the first balloon such that the first balloon at least partially inflates to substantially anchor the stent axially relative to the branch body lumen, and at least some of the inflation media passes through one or more openings in a membrane of the first balloon into an interior of the second balloon to inflate the second balloon to flare the first end of the stent within the ostium.

21. The method of claim 20, wherein delivering the inflation media causes the second balloon to inflate simultaneously with the first balloon.

22. The method of claim 21, wherein the one or more openings are configured such that delivering the inflation media causes the first balloon to inflate at a faster rate than the second balloon.

23. The method of claim 20, wherein the one or more openings are configured such that delivering the inflation media causes the first balloon to at least partially inflate before the second balloon begins to inflate.

24. The method of claim 20, wherein, if pressure of the inflation media delivered through the lumen exceeds a predetermined threshold, a pressure relief feature is activated to limit the pressure delivered into the interiors of the first and second balloons.
